(19) United States
(12) Patent Application Publication
(10) Pub. No.: US 2024/0336941 A1
(43) Pub. Date: Oct. 10, 2024

(54) METHODS FOR PRODUCING BIOGAS AND RENEWABLE NATURAL GAS USING CO-PRODUCTS FROM A STILLAGE STREAM AS FEEDSTOCKS

(71) Applicant: Marquis IP LLC, Hennepin, IL (US)

(72) Inventors: Jennifer Aurandt-Pilgrim, Hennepin, IL (US); Tyler Angus, Princeton, IL (US)

(21) Appl. No.: 18/628,548

(22) Filed: Apr. 5, 2024

Related U.S. Application Data
(60) Provisional application No. 63/457,778, filed on Apr. 6, 2023.

(51) Int. Cl. C12P 5/02; C05F 5/00; C10L 3/10

(52) U.S. Cl. CPC C12P 5/023; C05F 5/008; C10L 3/104; C10L 2290/26

(57) ABSTRACT

Disclosed herein are methods and systems for producing biogas and/or renewable natural gas. Methods of producing biogas and/or renewable natural gas include using clarified thin stillage, clarified thin stillage syrup, or any combination thereof, as a feedstock for anaerobic digestion to produce the biogas and/or renewable natural gas from the resultant biogas. Methods for producing biogas and/or renewable natural gas include: (a) introducing feedstock comprising clarified thin stillage or clarified thin stillage syrup to an anaerobic digester; and (b) digesting the feedstock to produce methane-containing gas streams, such as biogas and/or renewable natural gas.

### CROSS-REFERENCE TO RELATED APPLICATIONS

This application claims priority to U.S. Provisional Patent Application No. 63/457,778, filed Apr. 6, 2023, the entire contents of which are hereby incorporated by reference.

### BACKGROUND

Since the Renewable Fuel Standard mandated ethanol as the preferred oxygenate for blending with gasoline, the dry-grind ethanol industry in North America experienced tremendous growth. The mandate spurred extensive expansion of dry-grind ethanol production across the US Midwest grain belt, with peak production near 17.5 billion gallons per year from over 200 facilities. More recently, there has been a demand for low-carbon-intensity fuels and feedstocks that has increased the demand for ethanol and co-products from the corn-to-ethanol fermentation pathway. Additionally, producers seek to improve operating and energy efficiency and maximize the total value created from each bushel of grain (e.g., corn) processed through ethanol production. There are market incentives to decrease the carbon intensity of the ethanol process which can include the use of renewable natural gas. Thus, there exists significant commercial interest in methods that permit high-efficiency recovery of valuable co-products from stillage, including clarified thin stillage (CTS) and clarified thin stillage syrup, as well as production of renewable fuel streams (e.g., biogas, methane and/or renewable natural gas) from such co-products.

It is against this backdrop that the methods and systems of the present disclosure were developed.

### SUMMARY

In one aspect, which may be combined with any other aspect or embodiment, the present disclosure relates to a method for producing biogas, comprising: (a) introducing a feedstock comprising clarified thin stillage (CTS) or clarified thin stillage syrup (CTS syrup) to an anaerobic digester; and (b) digesting the feedstock in the anaerobic digester to produce biogas.

In another aspect, which may be combined with any other aspect or embodiment, the present disclosure relates to a method of producing renewable natural gas (RNG), the method comprising: (a) introducing a feedstock comprising clarified thin stillage (CTS) or clarified thin stillage syrup (CTS syrup) to an anaerobic digester; (b) digesting the feedstock to produce biogas; (c) removing CO₂ from the biogas to produce a CO₂-rich stream and a methane-rich stream; and (d) further purifying the methane-rich stream to produce renewable natural gas.

In some embodiments, the feedstock comprises CTS. In some embodiments, the CTS comprises total solids at a concentration of 3 wt. % to 5 wt. %, relative to the total weight of the CTS. In some embodiments, the CTS comprises total suspended solids at a concentration of 7 wt. % to 15 wt. %, relative to the total weight of solids in the CTS.

In some embodiments, the feedstock comprises CTS syrup. In some embodiments, the CTS syrup comprises 10 wt. % to 80 wt. % of total solids, relative to the total weight of the CTS syrup. In some embodiments, the CTS syrup comprises total suspended solids at a concentration of less than or equal to about 15 wt. %, relative to the total weight of solids in the CTS syrup. In some embodiments, the CTS syrup comprises total suspended solids at a concentration of 7 wt. % to 15 wt. %, relative to the total weight of solids in the CTS syrup.

In some embodiments, the feedstock comprises CTS and CTS syrup. In some embodiments, the feedstock further comprises at least one selected from: process condensate; thermal vapor recompression water; water from a CO₂ scrubber unit; or water from CO₂ compression.

In some embodiments, the digesting (b) is performed using a hydraulic retention time of 1 minute to 32 days. In some embodiments, the digesting (b) is performed using a hydraulic retention time of 16 days to 20 days. In some embodiments, the digesting (b) is performed using a solids retention time of at least 20 days. In some embodiments, the digesting (b) is performed using a solids retention time of 28 days to 32 days.

In some embodiments, the method comprises purifying the biogas to produce renewable natural gas (RNG). In some embodiments, the purifying comprises separating CO₂ from the biogas to produce a CO₂-rich stream and a methane-rich stream.

In some embodiments, the methane-rich stream is further purified to produce the renewable natural gas (RNG). In some embodiments, the method comprises blending the RNG with natural gas.

In some embodiments, the anaerobic digester is a high-rate reactor. In some embodiments, the anaerobic digester is a stirred tank reactor.

In some embodiments, the CTS or CTS syrup has a native C:N ratio. In some embodiments, an additive ingredient is added to the CTS or the CTS syrup to modify a C:N ratio.

In some embodiments, the method comprises removing residual biomass remaining in the anaerobic digester after (b). In some embodiments, the method comprises processing residual biomass remaining in the digester after (b) for use as a fertilizer. In some embodiments, the method comprises processing residual biomass remaining in the digester after (b) for use as single cell protein.

In some embodiments, the method comprises directing the biogas, methane-rich gas stream, or RNG to an upstream process as an energy source.

In some embodiments, the CO₂-rich stream is directed to a carbon capture and/or sequestration or utilization process.

In some embodiments, the method comprises blending the RNG with natural gas.

### BRIEF DESCRIPTION OF THE DRAWINGS

The Detailed Description is set forth with reference to the accompanying drawings. In the drawings, the use of the same reference numbers in different drawings or figures indicates similar or identical items. The features illustrated in the drawings are not necessarily drawn to scale, and features of one embodiment may be employed with other embodiments or features may not be employed in all embodiments as the skilled artisan would recognize, even if not explicitly stated herein.

FIG. 1 is a block flow diagram depicting a conventional dry-grind ethanol process.

FIG. 2 is a block flow diagram illustrating a method of recovering co-products (e.g., CTS and CTS syrup) from a stillage stream for use as feedstocks in an anaerobic digestion process, according to one exemplary embodiment.

FIG. 3 is a block flow diagram illustrating a method of recovering co-products (e.g., CTS and CTS syrup) from a stillage stream for use as feedstocks in an anaerobic digestion process, according to one exemplary embodiment.

FIG. 4 is a block flow diagram of a method for producing biogas or renewable natural gas via anaerobic digestion using clarified thin stillage (CTS), CTS syrup, or a combination thereof, as a feedstock for anaerobic digestion.

FIG. 5A is a plot of cumulative methane production versus time for an anaerobic digestion process using CTS as the feedstock,

FIG. 5B is a plot of cumulative biogas production versus time for an anaerobic digestion process using CTS as the feedstock.

FIG. 6A is a plot of cumulative methane production versus time for an anaerobic digestion process using CTS syrup as the feedstock.

FIG. 6B is a plot of cumulative biogas production versus time for an anaerobic digestion process using CTS syrup as the feedstock.

### DETAILED DESCRIPTION

### Definitions

For purposes of this application the definitions of these terms are as follows:

“Anaerobic digester” means a unit operation within which anaerobic fermentation occurs. Anaerobic digester can mean a number of different configurations or types of digesters, including for example continuously stirred tank reactor or high-rate reactors.

“Backset” means the portion of centrate, thin stillage or clarified thin stillage returned to the front of the plant and mixed with fresh ground grain.

“Biogas” means the gases which result from anaerobic fermentation of organic material. In some embodiments, biogas includes methane and carbon dioxide.

“Biochemical Oxygen Demand” (or BOD) means the amount of dissolved oxygen (DO) needed by aerobic biological organisms to break down organic material present in a given sample at a certain temperature over a specific time period.

“Centrate” means the portion of a post-fermentation spent grains process stream remaining after whole stillage has passed through a separating device such as a centrifuge and/or screens, wherein heavier wet cake solids have been removed.

“Clarified thin stillage” (or “CTS”) means a portion of the thin stillage stream from which 80% or more of the suspended solids have been removed.

“Coagulant” means a chemical, which induces coagulation, e.g., induces the initial agglomeration of material suspended within a liquid.

“Chemical Oxygen Demand” (or COD) means the amount of oxygen equivalents consumed in the chemical oxidation of organic matter by a strong oxidant (e.g., potassium permanganate).

“Crude protein” means the nutritionist's conventional approximation of actual protein content of a sample of feed, food, or other biological material, as determined by applying an appropriate factor to the sample's elemental nitrogen content. By convention, crude protein (wt. %) is calculated by multiplying elemental nitrogen (wt. %) by a factor of 6.25. Methods of determining elemental nitrogen include, e.g., combustion methods and wet-chemistry methods (e.g., Kjeldahl). Herein, the factor of 6.25 is used, unless otherwise indicated.

“Distillers Dried Grains” (or “DDG”) is the product obtained after the removal of ethyl alcohol by distillation from the yeast fermentation of a grain or grain mixture by separating the resultant coarse grain fraction of the whole stillage and drying it by methods employed in the grain distilling industry.

“DDGS” (or “distillers dried grains with solubles”) means the product obtained after the removal of ethyl alcohol by distillation from the yeast fermentation of a grain or grain mixture by condensing and drying at least ¾ of the solids of the resultant methods employed in the grain distilling industry (2021, AAFCO Official Publication, Chapter 6, Section 27.6)

“Displacement washing” means purifying a layer or bed of solids by displacing interstitial, absorbed or adsorbed particulates and liquids within the layer of solids with a liquid of higher purity. Displacement washing can be performed on a static or dynamic (moving) bed of solids. A pressure differential or centrifugal force may be applied to the solids to affect displacement washing.

“Dissolved solids” means matter other than water remaining in the collected filtrate of a liquid sample filtered through a nominal 0.22-μm pore-size membrane. Dissolved solids in the filtrate comprise soluble inorganic and organic compounds and very fine suspended solids.

“Downstream” and “upstream” mean process streams or unit operations downstream and upstream of distillation (ethanol removal), respectively. In a conventional dry-grind ethanol process, whole stillage is considered to be a “downstream” process stream.

“Flocculant” means a chemical which induces flocculation, e.g., it induces agglomeration of material suspended within a liquid either alone or after coagulation when the liquid is stirred or otherwise mixed.

“Grain” means plant seeds that provide the starch fraction required to produce fermentable sugars for the production of ethanol. Example grains include corn, wheat, rice, milo, sorghum, and barley. Grain-derived starch can be supplemented with simple sugars from sugar cane and sugar beets in the fermentation process.

“High-protein animal feed composition” (or “high-protein animal feed” or “high-protein animal feed ingredient”) means an animal feed composition derived from stillage solids and having at least about 40 wt. % of crude protein on a dry matter basis. For example, a high-protein animal feed composition may have protein at a concentration of at least about 40 wt. %, at least about 45 wt. %, at least about 50 wt. %, at least about 55 wt. %, at least about 60 wt. %, or greater, on a dry matter basis.

“Hydraulic Retention Time (HRT)” means the amount of time that the liquid portion of the digestate remains in a given portion of the anaerobic digester. Long HRTs allow more time for solids to separate from the liquid portion and to be digested by bacteria. Shorter HRTs reduce the ability for solids to separate from the liquid portion but allow for the use of smaller tanks because they require shorter HRTs require less water than longer HRTs, enabling a size reduction for the overall system.

“Natural gas” means a mixture of gases derived from petroleum, primarily composed of methane and other hydrocarbons, and may be in gaseous or liquid form. Natural gas can be naturally forming, or it can be synthesized. The quality of this gas is considered to be defined as the gas allowed in a natural gas supply chain. “Natural gas” may also be referred to as “pipeline-quality natural gas.”

“Renewable Natural Gas” (RNG) is a pipeline-quality bio-methane that is fully interchangeable with conventional natural gas. RNG is purified from biogas, which is formed from the decomposition of organic matter, often via anaerobic digestion. In some instances, “pipeline-quality bio-methane,” “pipeline-quality methane,” and “renewable natural gas (RNG)” are used interchangeably in this application.

“Oil” means grain-based oil such as corn oil. Oil present in centrate or thin stillage can be separated as a low gravity liquid from higher-gravity water and water-borne solids. The recovered oil of a dry-grind ethanol process is commonly referred to as distiller's corn oil (“DCO”).

“Protein concentrate” means a composition derived from stillage suspended solids and having greater than 50 wt. % (e.g., at least about 60 wt. %, at least about 65 wt. %, at least about 70 wt. %, at least about 75 wt. %, or greater) of crude protein on a dry matter basis.

“Single cell protein” means protein that originates from a single cell type, such as microbial protein, algal protein, fungal protein. Single cell protein can originate from unicellular organisms, such as bacteria, or from multicellular organisms, such as filamentous fungi.

“Solids” means the non-water portion that remains in stillage after distillation including germ, protein, gluten, hull, lipids, carbohydrates and spent yeast.

“Solids Retention Time (SRT)” is the amount of time that sludge and microbes remain in the anaerobic digester. As the digestate flows into a digestion vessel (e.g., the digester), some time is required for the solids to naturally precipitate from the liquid portion of the digestate. After the solids have separated, there is another amount of time required for microbes within the anaerobic digestion system to fully digest the solids and produce biogas.

“Stillage” means the solids present after distillation of an ethanol fermentation stream, commonly referred to as whole stillage and/or thin stillage either as generated in the process or in a concentrated form (e.g., additional water may be removed).

“Suspended solids” means all solid matter, other than water, retained on a nominal 0.22-μm pore-size filter. (See “dissolved solids”.) In the context of stillage, suspended solids comprise non-fermentable grain fragments (e.g., hull and fiber) and agglomerated macromolecules (e.g., proteins, lipids, cells, and cellular debris, etc.).

“Syrup” means the portion of thin stillage that has passed through a concentration or evaporation process and has reached the optimum solids level for process limitations (e.g., viscosity) in addition to an application to wet feed or DDGS drying operations. For instance, “clarified thin stillage syrup” or “CTS syrup” means a portion of clarified thin stillage that has passed through a concentration or evaporation process.

“Thermal Vapor Recompression (TVR)” means the compression of water vapor from an evaporator, which is entrained and compressed with higher-pressure steam and is subsequently condensed in an evaporator heat exchanger. The condensate from this process contains water and volatiles (e.g., organic acids) and may be added to clarified thin stillage or clarified thin stillage syrup as a source of digestible volatiles (e.g., organic acids) or secondary input for an anaerobic digestion system. TVR may be performed using a thermocompressor (e.g., a Dryer Emission Energy Recovery System (DEERS)).

“Thin Stillage” means the portion of a post-fermentation and distillation grains process stream which is evaporated to produce condensed distillers solubles or “syrup”.

“Washing” means a process wherein a wash liquid is added to a separated solids stream for the purpose of removing additional dissolved or suspended solids from the separated solids. The washing process yields washed, separated solids. Used wash liquid may be re-used in a multi-stage washing process or diverted to upstream or downstream process steps.

“Wet cake” (or “wet distillers grains” (“WDG”)) means the portion of stillage solids remaining after separation of a post-fermentation spent grains process stream by a separator (e.g., decanter centrifuge). In the context of this disclosure, “wet cake” may be used in reference to wet cake derived from whole stillage (as opposed to the whole stillage itself) or wet cake derived from flocculated suspended solids of centrate. “Wet cake derived from DAF solids” means the portion of solids remaining after separation of the flocculated solids stream discharged from a DAF by a separator (e.g., a decanter centrifuge).

“Whole stillage” means the portion of a post-fermentation and distillation grains process stream remaining after the spent grain-based material has passed through a distillation process by which ethanol has been removed.

Methods for Recovering Clarified Thin Stillage (CTS) and CTS Syrup from Stillage Streams

The methods described herein produce CTS and CTS syrup, among other co-products. The CTS and CTS syrup are in turn used in the presently disclosed methods as feedstocks production of methane-containing gas streams, such as biogas or renewable natural gas (RNG).

Without intending to be limited to any particular method or source of CTS or CTS syrup, the following disclosure describes methods for obtaining CTS and/or CTS syrup for use as feedstocks in the present technology. Such methods are described in, e.g., U.S. Pat. Nos. 9,776,105; 11,220,663; 11,359,218; U.S. Patent Appl. Publ. No. US 2022/0112450A1; U.S. Patent Appl. Publ. No. US 2022/0049184A1; and U.S. application Ser. No. 16/687,608, filed Nov. 18, 2019 (published as US 2020/0154730A1), the entireties of which are hereby incorporated by reference. Discussion of conventional dry-grind ethanol processes and methods for high-efficiency recovery of co-products (e.g., CTS and/or CTS syrup) from stillage streams are discussed below but are not intended to be limiting.

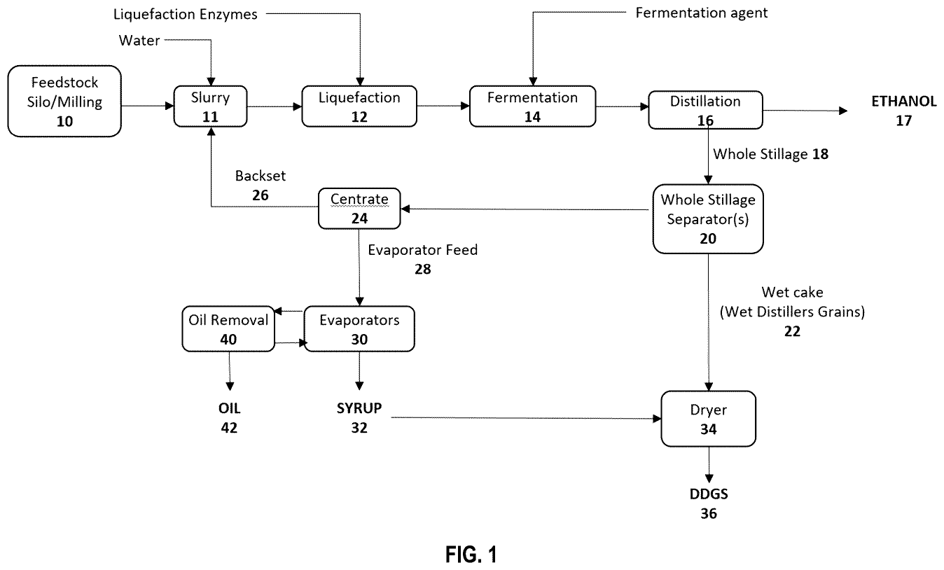

FIG. 1 shows a conventional process flow for a dry-grind ethanol process. Briefly, grain (e.g., corn) is ground 10, slurried with water and backset 11, and treated with heat and enzymes (e.g., alpha-amylase, glucoamylase, etc.) to liquefy and hydrolyze the available starch to simple fermentable sugars 12. A fermentation agent (e.g., yeast/bacteria) is added to convert (ferment) the sugars to ethanol 14. The fermented mash is distilled 16 to separate and recover ethanol. The residual aqueous slurry of spent grains (i.e., the whole stillage 18) contains fragments of germ, fiber, oil, unconverted starch, unfermented sugars, whole and degraded cells of the fermentation agent (e.g., yeast), off-product metabolites (e.g., glycerol and short chain organic acids), proteins of the grain and the fermentation agent, and other suspended and dissolved solids.

Referring still to FIG. 1, the whole stillage stream 18 is generally separated 20 into wet cake derived from whole stillage 22 and centrate 24. Whole stillage separation 20 may be performed by centrifugation, decantation, filtration, or any other suitable means. The wet cake 22 derived from whole stillage can be dried 34 to produce Dry Distillers Grains with Solubles (“DDGS”) 36. A portion of the centrate, referred to as backset 26, is recycled back to the front end of the ethanol process as make-up water. The remaining centrate (“evaporator feed”) 28 is fed to an evaporator 30 and concentrated to a syrup 32 comprising concentrated solubles and fine suspended solids. Distiller's corn oil (DCO) 42 can be separated from concentrated centrate or syrup 32 by, e.g., centrifugation (oil removal) 40 at the evaporator. Syrup 32, also known as condensed distiller's solubles (CDS) can be sold separately, but it is more typically added to the wet cake 22 derived from whole stillage and dried 34 to produce Dried Distillers Grains with Solubles (DDGS) 36.

Referring still to FIG. 1, wet cake 22 derived from whole stillage and DDGS 36 are valuable co-products that are important to the economic viability of conventional ethanol processes. For convenience, DDG, WDG, and DDGS are referred to as distiller's grain products. Typical commercial DDGS from a dry-grind corn ethanol plant contain about 28-30 wt. % crude protein, by weight of dry matter.

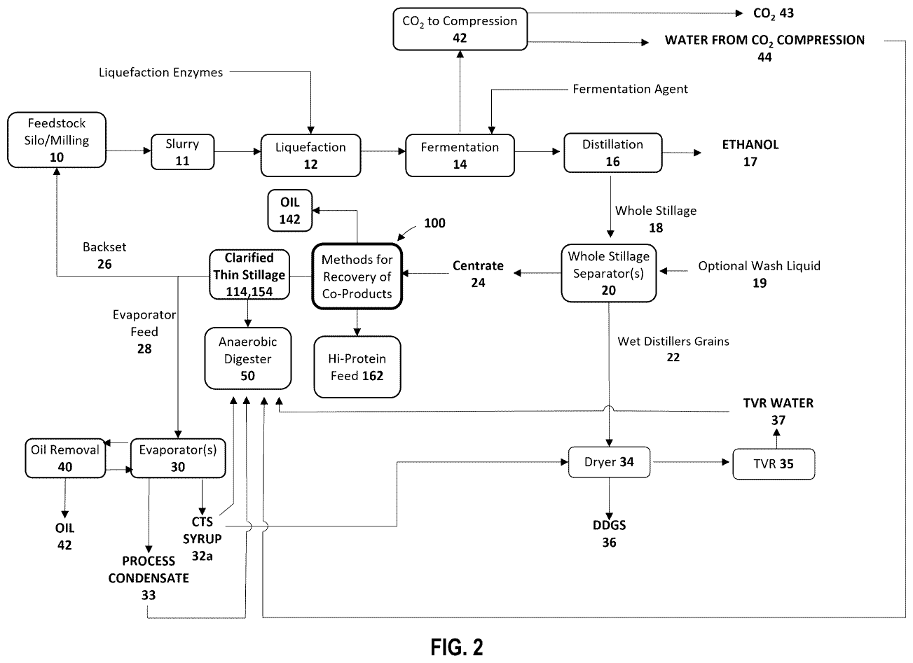

Referring now to FIG. 2, an exemplary method for recovering co-products (e.g., high-protein animal feed, CTS, and/or CTS syrup) from stillage streams is described in U.S. application Ser. No. 16/687,608, filed Nov. 18, 2019 (published as US 2020/0154730A1), the entirety of which is incorporated by reference herein.

As shown in FIG. 2, such methods 100 may be integrated into conventional dry-grind ethanol production processes. In such high-efficiency co-product recovery methods, whole stillage 18 is separated by one or more separators 20 to produce centrate 24 and wet cake 22 derived from whole stillage. The one or more separators 20 may be arranged in series, in parallel, or permutations thereof. By way of non-limiting example, the one or more separators 20 comprise(s) one or more horizontal decanter centrifuges (e.g., commercially available from Alfa Laval, GEA, Andritz, Flottweg, Centrisys and others), screens, sieves, filters, filtering centrifuges, and combinations thereof, including combinations including a decanter centrifuge. The one or more separator(s) may comprise one or more horizontal decanter centrifuges.

Referring still to FIG. 2, all or a portion of centrate 24 may be routed to co-product recovery method 100. For instance, some processes or ethanol plants may route a portion of untreated centrate to backset, for example, as a means of utilizing potential nutrients available in untreated centrate. Meanwhile, other systems and processes may route all of the centrate 24 to a co-product recovery method 100 as described herein.

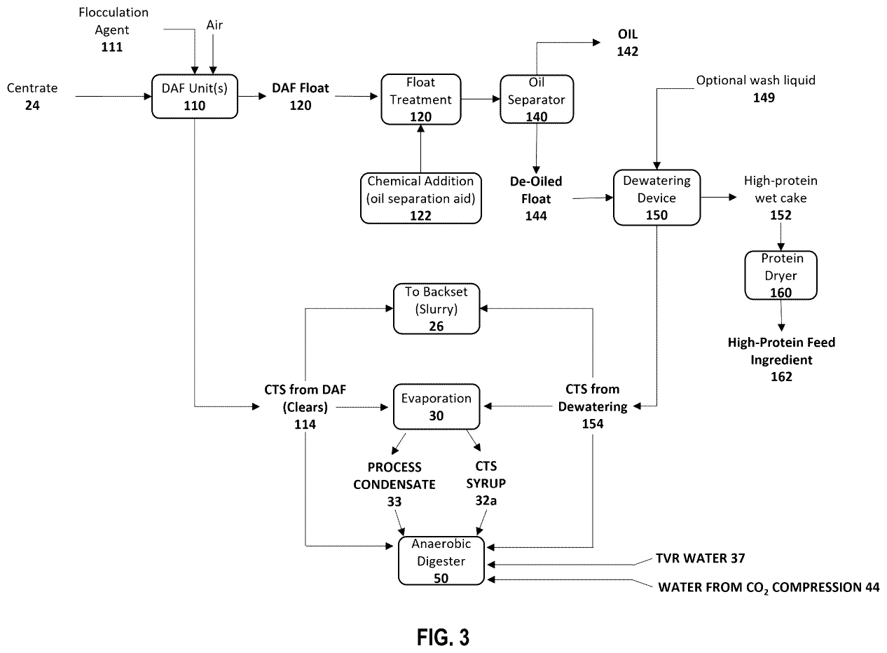

Referring to FIGS. 2-3, within co-product recovery methods 100 according to the present disclosure, suspended solids rich in oil and protein are removed from centrate 24 by a separator configured to receive a centrate stream 24 from a whole stillage separator 20 of a dry-grind ethanol plant, wherein the centrate stream 24 comprises soluble solids and insoluble solids, the soluble and insoluble solids comprising protein and oil. In some embodiments, the first separator is configured to separate a flocculated solids portion and a clarified thin stillage portion 114 from the centrate stream 24. In some embodiments, the separator is a dissolved air flotation (DAF) apparatus 110.

Referring still to FIGS. 2-3, co-product recovery methods of the present disclosure remove suspended solids from the centrate stream 24 to produce one or more clarified thin stillage (CTS) streams 114, 154. Any portion or all CTS produced in the co-product recovery method 100 may be routed to backset 26 and/or evaporators 30, as may be implemented in conventional dry-grind ethanol processes (see FIG. 2). Any or all CTS 114, 154 produced in the co-product recovery method 100 may be routed to an anaerobic digester 50 as feedstock for producing methane-containing gas streams (e.g., biogas and/or renewable natural gas).

Oil 42 may be removed 40 from CTS in a conventional evaporator oil removal process 40. Meanwhile, the suspended solids produced by the separator in the co-product recovery method 100 (e.g., by flotation in a DAF apparatus 110) are treated (e.g., by heating and/or addition of an oil extraction aid) to recover oil 142 from the suspended solids. The protein-rich heavy phase solids remaining after separation of oil 142 may be further treated (e.g., dewatered and dried) to produce a high-protein feed ingredient 162 having crude protein content greater than 45 wt. %, on a dry matter basis. This high-protein feed ingredient may be obtained by a method such as that disclosed in U.S. application Ser. No. 16/687,608, filed Nov. 18, 2019 (published as US 2020/0154730A1), the entirety of which is incorporated by reference herein.

Referring still to FIG. 3, in high-efficiency co-product recovery methods 100 that may produce CTS and/or CTS syrup as feedstocks, suspended solids rich in oil and protein are removed from centrate 24 by a first separator 110 configured to receive a centrate stream 24 from a whole stillage separator 20 of a dry-grind ethanol plant, wherein the centrate stream 24 comprises soluble solids and insoluble solids, the soluble and insoluble solids comprising protein and oil. CTS (“clears”) 114 streams and DAF float 112 streams from multiple DAF units may be combined into a single clears stream or a single float stream, respectively, for further downstream treatment or processing. The required number and dimensions of first separators 110 (e.g., DAF units) are established based on total volumetric flow (gallons per minute) of centrate 24, the buoyancy and rise rate of the flocs obtained therefrom, and desired clarity of underflow. Air is dissolved into the centrate under pressure in the DAF apparatus 110.

Referring still to FIG. 3, in some embodiments, the method further comprises contacting the centrate stream 24 with a flocculation agent (or “flocculant”) 111 to produce a flocculated solids portion (or “float” or “float layer” or “float solids”) 112 and a first clarified thin stillage (or “clears”) portion 114. The flocculation agent 111 is capable of bridging the suspended solids and causing them to “bloom” into large (1-10 mm or greater) agglomerates, or fluffy, low-density particle clusters known as “flocs.” Upon release of pressure, fine micro-bubbles of air form and rise. The flocs adhere to the micro-bubbles and are lifted to the DAF surface to form a “float” layer or flocculated solids portion 112, which is continuously removed from the surface of the DAF unit. Floc buoyancy is also enhanced by the presence of relatively low-density oil droplets in the suspended solids.

Flocculating agents comprise chemical coagulants and flocculants (and combinations thereof) known in the art. See generally THE NALCO WATER HANDBOOK, 2d Ed. (1988). In some embodiments, the flocculant comprises at least one selected from: anionic polymers; cationic polymers; non-ionic polymers; cationic coagulants; and combinations, copolymers, or mixtures thereof. In some embodiments, the flocculant comprises an anionic polymer or copolymer. In some embodiments, the flocculant is a high molecular weight anionic polyacrylamide. In some embodiments, the flocculant comprises an anionic copolymer of polyacrylamide and polyacrylic acid.

Further details regarding the DAF process are disclosed in U.S. application Ser. No. 16/687,608, filed Nov. 18, 2019 (published as US 2020/0154730A1), the entirety of which is incorporated by reference herein.

By virtue of suspended solids flocculation, the concentration of total solids in DAF float (or flocculated solids portion) 112 may be about 18 wt. % to about 50 wt. %, or about 20 wt. % to about 40 wt. % wt. % (e.g., about 19 wt. %, about 20 wt. %, about 21 wt. %, about 22 wt. %, about 23 wt. %, about 24 wt. %, or about 25 wt. %) as compared with 5-8 wt. % total solids in the incoming centrate 24.

In some embodiments, a method according to the present disclosure further comprises treating the flocculated solids portion 112 to produce a treated flocculated solids portion. DAF float 112 is pumpable and is forwarded to treatment system 120 comprising means for heating, adding an oil extraction aid, and aging.

Referring still to FIG. 3, in some embodiments, a method according to the present disclosure further comprises separating the treated flocculated solids portion (or “DAF float”) 112 to produce a light phase portion comprising oil 142 and a heavy phase portion comprising water and heavy phase solids, wherein the heavy phase solids comprise protein. In some embodiments, the separating 140 is performed using a high-speed disc centrifuge (HSDC) or any other suitable means of separating the light phase comprising oil 142 from the heavy phase comprising protein. In some embodiments, DAF float treatment improves the release of oil 142 in centrifugal oil separator 140.

In some embodiments, the heavy phase from separator 140 is referred to as de-oiled float 144 and contains greater than 40 wt. % crude protein on a dry matter basis. In some embodiments, the heavy phase contains from about 35 wt. % to about 60 wt. % or from about 40 wt. % to about 55 wt. % crude protein on a dry matter basis.

Referring still to FIG. 3, in some embodiments, co-product recovery methods 100 according to the present disclosure further comprise dewatering 150 the heavy phase portion (the de-oiled float) to produce dewatered protein wet cake 152 and a second clarified thin stillage portion 154. Additional water is removed from de-oiled float 144 with a dewatering device 150. Soluble solids are also removed with water in device 150, contributing to an increase of protein in the dewatered high-protein wet cake 152 to greater than 35 wt. %, greater than 40 wt. %, or greater than 45 wt. % on a dry matter basis (e.g., greater than or equal to about 45 wt. %, greater than or equal to about 50 wt. %, greater than or equal to about 55 wt. %, greater than or equal to about 60 wt. %, greater than or equal to about 65 wt. %, greater than or equal to about 70 wt. %, greater than or equal to about 75 wt. %, or any range or value therein between). In some embodiments the dewatering device 150 is a high-speed horizontal decanter centrifuge. In some embodiments, the dewatering device 150 is a paddle screen. In some embodiments, the dewatering device 150 is a multiple-discharge type paddle screen. It has been found that dewatering effectiveness may be enhanced by addition of a flocculation aid 156 to the de-oiled float.

Referring still to FIG. 3, in some embodiments, co-product recovery methods 100 according to the present disclosure further comprise drying 160 the dewatered protein wet cake 152 to produce a high-protein animal feed ingredient 162.

As shown in FIG. 3, removal of suspended solids by methods according to the present disclosure results in one or more clarified thin stillage (CTS) streams 114, 154. CTS contains less than 30% (e.g., less than 20%) of the total suspended solids (e.g., about 19%, 18%, 17%, 16%, 15%, 14%, 13%, 12%, 11%, 10%, 9%, 8%, 7%, 6%, 5%, 4%, 3%, 2%, 1%, 0.5%, 0.1%, or less) present in the centrate feed stream 24. For instance, a first CTS stream 114 may be produced as DAF underflow 114, also known as “clears”. A second CTS stream 154 results from dewatering of the high-protein solids. Any or all CTS 114, 154 produced in the present methods 100 may be routed to backset 26, an anaerobic digester 50, and/or evaporators 30 as desired, based on the requirements of particular processes.

Referring to FIG. 3, in some embodiments, the centrate from the dewatering centrifuge 150 is a clarified thin stillage (CTS) stream 154. In some embodiments, CTS 154 from dewatering 150 is directed to upstream points in the process. In some embodiments, the CTS 154 may be combined with DAF clears (the first clarified thin stillage portion or CTS from DAF) 114 and/or any portions of centrate or thin stillage not treated by the process disclosed herein. In some embodiments, the CTS 154 from protein dewatering 150 is directed separately or in combination with DAF clears 114 and untreated thin stillage to a multi-stage evaporator system 30 and is concentrated to CTS syrup 32a. In some embodiments, CTS 154 from protein dewatering 150 is used as all or a portion of backset 26. In some embodiments, CTS 154 from protein dewatering is routed to an anaerobic digester 50 as a feedstock.

### Clarified Thin Stillage (CTS) and CTS Syrup Disposition

In some embodiments, multiple clarified thin stillage (CTS) streams are produced. In some embodiments, the primary CTS streams are DAF clears (DAF underflow) 114 and a CTS stream 154 produced by dewatering de-oiled DAF float 144. Secondary CTS streams may be derived if a primary CTS stream is subjected to further separation steps to remove additional suspended or dissolved solids. Any CTS stream, including such secondary CTS streams, may be routed to an anaerobic digester 50 as a feedstock. For example, additional suspended solids may be removed from DAF clears 114 by a second flocculation and second separation step (e.g., second flotation step, centrifugation, membrane concentration, or other solids separation methods known to those skilled in the art). In some embodiments, total suspended solids in any primary or secondary CTS stream are 90% or less (e.g., 75% or less, 70% or less, 65% or less, 60% or less, 55% or less, 50% or less, 45% or less, 40% or less, 35% or less, 30% or less, or 25% or less) by weight of centrate.

In some embodiments, any portion or all of the CTS 114, 154 and/or of a secondary CTS stream is directed to a separate process. In some embodiments, CTS 114 or 154 is directed to an anaerobic digester 50 for the production of biogas and/or renewable natural gas. In some embodiments, CTS syrup 32a is directed to an anaerobic digester 50 for production of biogas and/or renewable natural gas.

As shown in FIGS. 2-3, the CTS 114, 154, the CTS syrup 32a, or any combination thereof may be directed to an anaerobic digester 50 as feedstock for an anaerobic digestion process. Further product streams from the dry grind ethanol process may be added to the feedstock comprising CTS and/or CTS syrup, as “secondary feedstocks.” For instance, CO₂ from fermentation 14 may be purified and compressed 42 to recover purified CO2 43 and water from CO₂ compression 44, which may be added to the feedstock for an anaerobic digester 50. As another example, process condensate 33 (condensed water) from an evaporation system 30, such as that used to produce CTS syrup 32a, may be added to the feedstock for an anaerobic digester 50. As yet another example, TVR water 37 from thermal vapor recompression 35 (e.g., at dryer 34) may be added to the feedstock for an anaerobic digester 50.

Methods for Producing Biogas and/or Renewable Natural Gas Using CTS and/or CTS Syrup as a Feedstock for Anaerobic Digestion

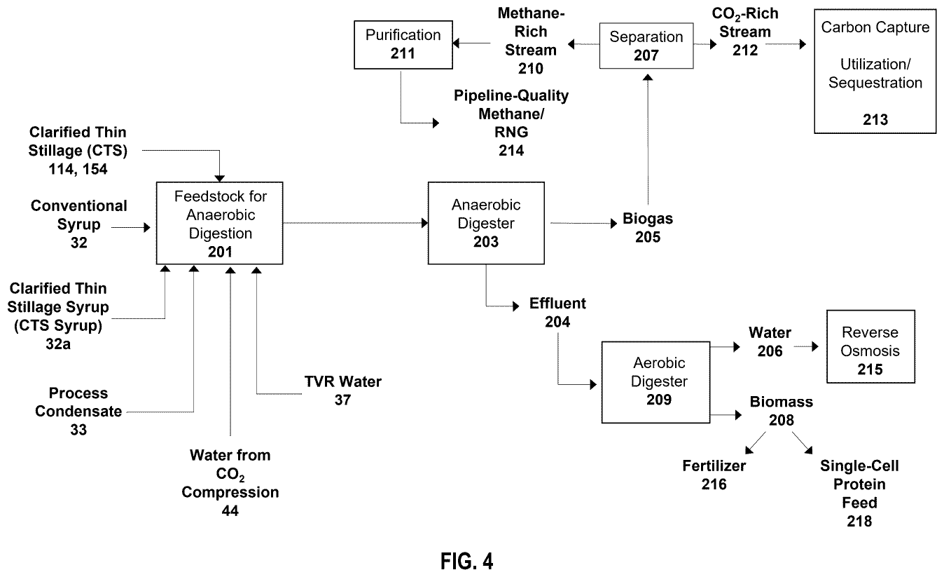

Referring now to FIG. 4, methods for producing biogas and/or renewable natural gas according to the present disclosure comprise: (a) introducing a feedstock 201 comprising clarified thin stillage, clarified thin stillage syrup, or a combination thereof, to an anaerobic digester 203; and (b) digesting the feedstock to produce biogas 205 and, in some embodiments, purifying the biogas to produce renewable natural gas.

### CTS as Feedstock

Although conventional clarified thin stillage (CTS) (obtained by methods disclosed in, e.g., U.S. Pat. Nos. 9,776,105; 11,220,663; 11,359,218; U.S. Patent Appl. Publ. No. US 2022/0112450A1; U.S. Patent Appl. Publ. No. US 2022/0049184A1; and similar methods) may be used as a feedstock in the present methods, in some embodiments, the CTS is obtained by a method such as those described in, e.g., U.S. application Ser. No. 16/687,608, filed Nov. 18, 2019 (published as US 2020/0154730A1). In such embodiments, methods according to the present disclosure use a clarified thin stillage (CTS) that includes very low concentrations of suspended solids (e.g., all or nearly all of the total solids are dissolved, as opposed to suspended).

In some embodiments, the clarified thin stillage (CTS) produced by the methods described above comprises solids (total solids) at a concentration of less than or equal to 20 wt. %, less than or equal to 15 wt. %, less than or equal to 10 wt. %, less than or equal to 9.5 wt. %, less than or equal to 9 wt. %, less than or equal to 8.5 wt. %, less than or equal to 8 wt. %, less than or equal to 7.5 wt. %, less than or equal to 7 wt. %, less than or equal to 6.5 wt. %, less than or equal to 6 wt. %, less than or equal to 5.5 wt. %, less than or equal to 5 wt. %, less than or equal to 4.5 wt. %, less than or equal to 4 wt. %, less than or equal to 3.5 wt. %, less than or equal to 3 wt. %, less than or equal to 2.5 wt. %, less than or equal to 2 wt. %, less than or equal to 1.5 wt. %, less than or equal to 1 wt. %, or less, or any range or value therein between, such as about 1 to 10 wt. %, about 2 to 8 wt. %, or about 3 to 5 wt. %.

In some embodiments, the clarified thin stillage comprises a very low concentration of suspended solids or no suspended solids. In some embodiments, the clarified thin stillage comprises suspended solids at a concentration of 90% % or less of the total solids, 25% or less of the total solids, 20% or less of the total solids, 15% or less of the total solids, 14% or less of the total solids, 13% or less of the total solids, 12% or less of the total solids, 10% or less of the total solids, 9% or less of the total solids, 8% or less of the total solids, 7% or less of the total solids, 6% or less of the total solids, 5% or less of the total solids, 4% or less of the total solids, 3% or less of the total solids, 2% or less of the total solids, 1% or less of the total solids, 0.5% or less of the total solids, 0.1% or less of the total solids, or 0% of the total solids, or any range or value therein between, such as about 1% to 20% of total solids, about 3% to 18% of total solids, about 5% to 15% of total solids, or about 8% to 12% of total solids By way of example, if the clarified thin stillage comprises 3 wt. % total solids, and the suspended solids are at a concentration of 10% of the total solids, the overall concentration of suspended solids would be 0.3 wt. %.

In some embodiments, the clarified thin stillage comprises dissolved solids at a concentration of 70% or more of the total solids, 71% or more of the total solids, 72% or more of the total solids, 73% or more of the total solids, 74% or more of the total solids, 75% or more of the total solids, 76% or more of the total solids, 77% or more of the total solids, 78% or more of the total solids, 79% or more of the total solids, 80% or more of the total solids, 81% or more of the total solids, 82% or more of the total solids, 83% or more of the total solids, 84% or more of the total solids, 85% or more of the total solids, 86% or more of the total solids, 87% or more of the total solids, 88% or more of the total solids, 89% or more of the total solids, 90% or more of the total solids, 91% or more of the total solids, 92% or more of the total solids, 93% or more of the total solids, 94% or more of the total solids, 95% or more of the total solids, 96% or more of the total solids, 97% or more of the total solids, 98% or more of the total solids, 99% or more of the total solids, 99.5% or more of the total solids, 99.9% or more of the total solids, or 100% of the total solids, or any range or value therein between, such as about 80% to 99% of total solids, about 82% to 97% of total solids, about 85% to 95% of total solids, or about 88% to 92% of total solids By way of example, if the clarified thin stillage comprises 3 wt. % total solids, and the dissolved solids are at a concentration of 90% of the total solids, the overall concentration of dissolved solids would be 2.7 wt. %.

### Conventional “Syrup” or CTS Syrup as Feedstock

In some embodiments, methods according to the present disclosure use a clarified thin stillage syrup or conventional syrup (hereinafter referred to, collectively, as “syrup”) as a feedstock for anaerobic digestion. In some embodiments, the present methods use CTS syrup that includes very low concentrations of suspended solids (e.g., all or nearly all of the total solids are dissolved, as opposed to suspended).

Although conventional syrup (obtained by methods disclosed in, e.g., U.S. Pat. Nos. 9,776,105; 11,220,663; 11,359,218; U.S. Patent Appl. Publ. No. US 2022/0112450A1; U.S. Patent Appl. Publ. No. US 2022/0049184A1; and similar methods) may be used as a feedstock in the present methods, in some embodiments, the CTS syrup is obtained by a method such as those described in, e.g., U.S. application Ser. No. 16/687,608, filed Nov. 18, 2019 (published as US 2020/0154730A1).

In some embodiments, the syrup (conventional syrup or CTS syrup) comprises solids (total solids) at a concentration of less than or equal to 80 wt. %, less than or equal to 75 wt. %, less than or equal to 70 wt. %, less than or equal to 65 wt. %, less than or equal to 60 wt. %, less than or equal to 57.5 wt. %, less than or equal to 55 wt. %, less than or equal to 52.5 wt. %, less than or equal to 50 wt. %, less than or equal to 47.5 wt. %, less than or equal to 45 wt. %, less than or equal to 42.5 wt. %, less than or equal to 40 wt. %, less than or equal to 37.5 wt. %, less than or equal to 35 wt. %, less than or equal to 32.5 wt. %, less than or equal to 30 wt. %, or less, or any range or value therein between.

In some embodiments, the CTS syrup comprises a very low concentration of suspended solids or no suspended solids. In some embodiments, the clarified thin stillage syrup comprises suspended solids at a concentration of 30% or less of the total solids 25% or less of the total solids, 20% or less of the total solids, 15% or less of the total solids, 14% or less of the total solids, 13% or less of the total solids, 12% or less of the total solids, 10% or less of the total solids, 9% or less of the total solids, 8% or less of the total solids, 7% or less of the total solids, 6% or less of the total solids, 5% or less of the total solids, 4% or less of the total solids, 3% or less of the total solids, 2% or less of the total solids, 1% or less of the total solids, 0.5% or less of the total solids, 0.1% or less of the total solids, or 0% of the total solids, or any range or value therein between. By way of example, if the syrup (conventional syrup or CTS syrup) comprises 50 wt. % total solids, and the suspended solids are at a concentration of 10% of the total solids, the overall concentration of suspended solids would be 5 wt. %.

In some embodiments, the syrup (conventional syrup or CTS syrup) comprises dissolved solids at a concentration of 70% or more of the total solids, 71% or more of the total solids, 72% or more of the total solids, 73% or more of the total solids, 74% or more of the total solids, 75% or more of the total solids, 76% or more of the total solids, 77% or more of the total solids, 78% or more of the total solids, 79% or more of the total solids, 80% or more of the total solids, 81% or more of the total solids, 82% or more of the total solids, 83% or more of the total solids, 84% or more of the total solids, 85% or more of the total solids, 86% or more of the total solids, 87% or more of the total solids, 88% or more of the total solids, 89% or more of the total solids, 90% or more of the total solids, 91% or more of the total solids, 92% or more of the total solids, 93% or more of the total solids, 94% or more of the total solids, 95% or more of the total solids, 96% or more of the total solids, 97% or more of the total solids, 98% or more of the total solids, 99% or more of the total solids, 99.5% or more of the total solids, 99.9% or more of the total solids, or 100% of the total solids, or any range or value therein between. By way of example, if the syrup (conventional syrup or CTS syrup) comprises 50 wt. % total solids, and the dissolved solids are at a concentration of 85% of the total solids, the overall concentration of dissolved solids would be 42.5 wt. %.

In some embodiments, the clarified thin stillage syrup used in the present methods contains much higher amounts of total solids (e.g., 40 wt. % or more) than conventional syrup produced by conventional methods (about 30 wt. % total solids or less) and much less suspended solids and more dissolved solids by concentration than clarified thin stillage syrup produced by conventional methods. Conventional syrup may contain, e.g., 30 wt. % total solids, of which 50% (25 wt. % overall) is dissolved and 50% (25 wt. % overall) is suspended. CTS syrup according to the present disclosure may contain, e.g., 30 wt. % of total solids, of which 85% to 95% (25.5 wt. % to 28.5 wt. % overall) is dissolved and 5% to 15% (1.5 wt. % to 4.5 wt. % overall) is suspended.

In an embodiment, the CTS syrup has a composition similar or equal to the composition shown in Table 1.

TABLE 1
Proximate Analysis of CTS Syrup (% of Dry Matter)

| Component | Wt. % (Dry Matter Basis) |
|---|---|
| Dry Matter (DM) | 50.47 |
| Dissolved Solids (% of DM) | 85.56 |
| Suspended Solids (% of DM) | 14.44 |
| Crude Protein | 22.40 |
| Fat (Ether Extract) | 0.79 |
| Crude Fiber (CF) | 35.58 |
| Neutral Detergent Fiber (NDF) | 0.02 |
| Acid Detergent Fiber (ADF) | 0.24 |
| Starch | 3.77 |
| Ash | 9.11 |

### Secondary Feedstocks

Additional (secondary) feedstocks may be added to the feedstock comprising CTS and/or CTS syrup for anaerobic digestion. In some embodiments, the secondary feedstocks may function as sources of digestible volatile compounds. Referring to FIGS. 2-4, in some embodiments, the secondary feedstocks may comprise one or more selected from: process condensate 33; water from CO₂ purification and compression 44; and water from thermal vapor recompression 37. Process condensate 33 may comprise water condensed within an evaporation system (e.g., evaporators 30 used to concentrate clarified thin stillage 114, 154 into CTS syrup 32). Water from thermal vapor recompression 37 may be obtained by condensing steam obtained in a dryer system 34 using a thermal recompressor 35 and routing the water condensed from that process into an anaerobic digester 50. Condensed water from CO₂ compression 44 (e.g., obtained from purifying and compressing CO2 42 from fermentation 14) may also be collected and routed to an anaerobic digester 50. These secondary feedstocks may comprise water and small (no greater than 500 ppm) amounts of volatile organic compounds (e.g., formaldehyde, methanol, etc.) or acids (e.g., lactic acid, formic acid, etc.).

The CTS, CTS syrup, and secondary feedstocks may be mixed together in a tank prior to introducing the feedstock (including CTS, CTS syrup, and secondary feedstocks) into the anaerobic digester 203.

### Anaerobic Digestion

In some embodiments, the present disclosure relates to the production of biogas or renewable natural gas via anaerobic digestion. In some embodiments, the anaerobic digestion occurs with no oxygen present. In some embodiments, the anaerobic digestion occurs with essentially no oxygen present (e.g., less than 5 vol. %, less than 4 vol. %, less than 3 vol. %, less than 2 vol. %, less than 1 vol. %, less than 0.5 vol. %, less than 0.2 vol. %, less than 0.1 vol. %, less than 0.05 vol. %, less than 0.01 vol. %, or any range or value therein between).

In some embodiments, the anaerobic digestion can is performed using an anaerobic digester. In some embodiments, the anaerobic digester has a single digestion chamber. In some embodiments, the anaerobic digester has multiple digestion chambers. In some embodiments, the anaerobic digester is a passive system, such as a covered lagoon. In some embodiments, the anaerobic digester is a low-rate system or a high-rate system.

In some embodiments, the anaerobic digester is a batch laded digester. In some embodiments the anaerobic digester is a continuous flow digester. In some embodiments, the anaerobic digester maintains constant or varying conditions while anaerobic digestion occurs, including temperature, motion, pH and air pressure. In some embodiments, the anaerobic digester is a high-rate reactor (e.g., a high-rate reactor) will be known to the person having skill in the art.

Referring back to FIG. 4, in some embodiments, the anaerobic digester includes an outlet for removing cumulative biogases. In some embodiments, the anaerobic digester includes an outlet for removing residual biomass 208. In some embodiments, the anaerobic digester includes an outlet for effluent discharge 204, which may be routed to an optional aerobic digester 209. Water 206 from digestion may purified (e.g., by reverse osmosis 215) and recycled back into the dry grind ethanol process (e.g., into backset 26 or upstream processes (e.g., slurry 11)). In some embodiments, the effluent discharge is returned to the digester or removed for downstream purposes. In some embodiments, the residual biomass 208 is returned to the digester or removed for downstream purposes (e.g., use as fertilizer 216 or single-cell protein feed 218).

In some embodiments, anaerobic digestion includes hydrolysis, acidogenesis, acetogenesis, and methanogenesis, occurring simultaneously, sequentially, or some combination thereof. In some embodiments, the anaerobic digester receives an inoculum of at least one bacteria strain, optionally with organic matter to allow anaerobic digestion to begin growing via a propagation stage. In some embodiments, the inoculum propagation growth stage results in endogenous methane production which can be determined relative to methane production from digestion of a provided feedstock. In some embodiments, the methane produced from a provided feedstock exceeds the endogenous methane produced from the propagation stage.

In some embodiments, the anaerobic digester receives a feedstock of organic matter at a rate of up to 10 kg of volatile solids per cubic meter of digester capacity per day, which is then anaerobically digested. In some embodiments, the anaerobic digester receives a feedstock at a rate of up to 10 kg, 9 kg, 8 kg, 7 kg, 6 kg, 5 kg, 4 kg, 3 kg, 2 kg, 1 kg, 0.9 kg, 0.8 kg, 0.7 kg, 0.6 kg, 0.5 kg, 0.4 kg, 0.3 kg, or 0.2 kg per day (such as about 0.2 kg/day to about 10 kg/day, about 0.5 kg/day to about 8 kg/day, or about 1 kg/day to about 5 kg/day). In some embodiments, the feedstock is a co-product of a dry grind ethanol fermentation process, including, for example, clarified thin stillage, conventional syrup, clarified thin stillage syrup, “secondary feedstocks” (e.g., process condensate, TVR water, water from CO₂ purification and compression, etc.), or any combination thereof.

In some embodiments, the anaerobic digestion uses one or more bacterial strains to accomplish digestion. In some embodiments, the bacterial strains may include a thermophilic strain, a mesophilic strain, or a combination thereof.

In some embodiments, the anaerobic digestion occurs for a fixed duration. In some embodiments, the fixed duration is about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days. about 29 days, about 30 days, about 31 days, or about 32 days.

In some embodiments, the anaerobic digestion occurs at a fixed temperature effective to enable anaerobic digestion of a feedstock to product methane and/or natural gas at sufficient rate. In some embodiments, the fixed temperature is set at about 90° F., about 91° F., about 92° F., about 93° F., about 94° F., about 95° F., about 96° F., about 97° F., about 98° F., about 99° C., about 100° F., about 101° F., about 102° F., about 103° F., about 104° F., about 105° F., about 106° F., about 107° F., about 108° F., about 109° F., about 110° F., about 111° F., about 112° F., about 113° F., about 114° F., about 115° F., about 116° F., about 117° F., about 118° F., about 119° F., about 120° F., about 121° F., about 122° F., about 123° F., about 124° F., about 125° F., about 126° F., about 127° F., about 128° F., about 129° F., about 130° F., about 131° F., about 132° F., about 133° F., about 134° F., about 135° F., about 136° F., about 137° F., about 138° F., about 139° F., or about 140° F. In some embodiments, the anaerobic digestion occurs at a range of temperatures. In some embodiments, the anaerobic digestion occurs at a range of temperatures of about 90-100° F. In some embodiments, the anaerobic digestion is a mesophilic anaerobic digestion and occurs at a range of temperatures of about 90-100° F. (e.g., about 97° F.). In some embodiments, the anaerobic digestion is a thermophilic anaerobic digestion and occurs at a range of temperatures of about 125° F. to about 145° F. (e.g., about 135° F.).

In some embodiments, the pH of the anaerobic digestion will vary as digestion progresses. In some embodiments, initial pH will be lower than the pH when digestion is occurring at the peak rate. In some embodiments, pH will range from about 5.5-8.0 throughout the digestion process (e.g., about 5.5, about 6, about 6.5, about 7, about 7.5, or about 8, or any range or value therein between).

In some embodiments, the anaerobic digestion has a hydraulic retention time (HRT) suitable to achieve complete digestion of the feedstock. In some embodiments, the HRT is in a range of about 1 minute to about 32 days, about 1 hour to about 32 days, about 1 day to about 32 days, about 5 days to about 32 days, about 10 days to about 32 days, about 10 days to about 30 days, about 12 days to about 28 days, about 15 days to about 25 days, about 16 days to about 20 days, or any range or value therein between. In some embodiments, the HRT is less than or equal to about 32 days, less than or equal to about 30 days, less than or equal to about 25 days, less than or equal to about 20 days, less than or equal to about 19 days, less than or equal to about 18 days, less than or equal to about 17 days, less than or equal to about 16 days, less than or equal to about 15 days, less than or equal to about 12 days, less than or equal to about 10 days, less than or equal to about 5 days, less than or equal to about 4 days, less than or equal to about 3 days, less than or equal to about 2 days, less than or equal to about 1 day, less than or equal to about 20 hours, less than or equal to about 18 hours, less than or equal to about 16 hours, less than or equal to about 14 hours, less than or equal to about 12 hours, less than or equal to about 10 hours, less than or equal to about 8 hours, less than or equal to about 6 hours, less than or equal to about 4 hours, less than or equal to about 2 hours, less than or equal to about 1 hour less than or equal to about 50 minutes, less than or equal to about 40 minutes, less than or equal to about 30 minutes, less than or equal to about 20 minutes, less than or equal to about 10 minutes, less than or equal to about 5 minutes, less than or equal to about 1 minute, or any range or value therein between. In some embodiments, the HRT is about 1 minute to about 32 days. In some embodiments, the HRT is about 16 days to about 20 days. In some embodiments, the HRT is about 18 days.

In some embodiments, the anaerobic digestion has a solids retention time (SRT) suitable to achieve complete digestion of the feedstock. In some embodiments, the SRT is in a range of about 20 to about 50 days, about 25 to about 40 days, about 25 to about 35 days, about 28 to about 32 days, or any range or value therein between. In some embodiments, the SRT is less than or equal to about 50 days, less than or equal to about 45 days, less than or equal to about 40 days, less than or equal to about 35 days, less than or equal to about 30 days, less than or equal to about 28 days, less than or equal to about 25 days, or any range or value therein between. In some embodiments, the SRT is at least about 20 days. In some embodiments, the SRT is about 30 days.

In some embodiments, the carbon to nitrogen (C:N) ratio of the anerobic digestion feedstock is optimized to increase microbial activity without the use of carbon or nitrogen from sources other than the CTS or CTS syrup (e.g., CTS or CTS syrup produced according to conventional methods or according to the methods described in, e.g., U.S. application Ser. No. 16/687,608, filed Nov. 18, 2019 (published as US 2020/0154730A1). In some embodiments, the rate of growth of colony forming units of biogas-producing bacteria is affected by the C:N ratio of the anerobic digestion feedstock. In some embodiments the C:N ratio (native or modified) is below 40:1 (e.g., less than or equal to about 40:1, less than or equal to about 35:1, less than or equal to about 30:1, less than or equal to about 25:1, less than or equal to about 20:1, less than or equal to about 15:1, less than or equal to about 10:1, less than or equal to or about 5:1, or any range or value including and/or in between any two of these values). In some embodiments, the C:N ratio (native or modified) is at least 2:1, at least 3:1, at least 4:1, at least 5:1, at least 6:1, at least 7:1, at least 8:1, at least 9:1, at least 10:1, at least 12:1, at least 15:1, at least 20:1, or any range or value including and/or in between any two of these values.

In some embodiments, the C:N ratio is a “native ratio.” As used herein, the term “native ratio” or “native C:N ratio” means that no additives are provided to the feedstock to modify the C:N ratio. Rather, the C:N ratio is determined by the C:N ratio of the CTS or CTS syrup, without altering the feedstocks provided by the process of making CTS (e.g., from a conventional method; or from a DAF unit after the DAF float is removed, as shown in FIG. 3; or from a dewatering device after dewatering de-oiled float, as shown in FIG. 3) or of the CTS syrup (e.g., as produced by a conventional method; or as collected from an evaporation system, as shown in FIG. 3). In some embodiments, the C:N ratio is not a native ratio. In some embodiments, the C:N ratio is modified by addition of compounds capable of adjusting the C:N ratio (e.g., urea, ammonium nitrate, etc.) to the feedstocks provided by the process of making CTS (e.g., from a conventional method; or from a DAF unit after the DAF float is removed, as shown in FIG. 3; or from a dewatering device after dewatering de-oiled float, as shown in FIG. 3) or of the CTS syrup (e.g., as produced by a conventional method; or as collected from an evaporation system, as shown in FIG. 3).

In some embodiments, the anaerobic digestion uses CTS as feedstock. By way of non-limiting example, the CTS feedstock may have a composition similar to that shown in Table 3: about 46,947 mg/kg total solids, about 38,207 mg/kg volatile solids, about 1.88% protein content, about 95.24% moisture content, about 0.36% fat content, about 0.87% ash content, about 1.65% carbohydrate content, about 1.91% total organic carbon content, about 0.30% total nitrogen content, about 0.06% total sulfur content, about 5.6:1 C:N ratio, about 20,000 mg/L COD, about 50,000 mg/L COD, and a pH of about 4.47.

In some embodiments, the anaerobic digestion uses CTS syrup as feedstock. By way of non-limiting example, the CTS syrup feedstock may have a composition similar to that shown in Table 4: about 481,263 mg/kg total solids, about 401,461 mg/kg volatile solids, about 10.69% protein content, about 53.49% moisture content, about 3.61% fat content, about 7.98% ash content, about 24.23% carbohydrate content, about 20.07% total organic carbon content, about 1.71% total nitrogen content, about 0.52% total sulfur content, about 10:1 C:N ratio, about 200,000 mg/L BOD, about 550,000 mg/L COD, and a pH of about 4.25.

The anaerobic digestion produces biogas. In some embodiments, the biogas comprises methane and carbon dioxide. In some embodiments, the biogas comprises about 50-70 vol. % methane. In some embodiments, the biogas comprises about 30-50 vol. % carbon dioxide. In some embodiments the biogas includes about 50 vol. % methane and about 50 vol. % carbon dioxide. In some embodiments the biogas will include about 51 vol. % methane and about 49 vol. % carbon dioxide. In some embodiments the biogas will include about 52 vol. % methane and about 48 vol. % carbon dioxide. In some embodiments the biogas will include about 53 vol. % methane and about 47 vol. % carbon dioxide. In some embodiments the biogas will include about 54 vol. % methane and about 46 vol. % carbon dioxide. In some embodiments the biogas will include about 55 vol. % methane and about 45 vol. % carbon dioxide. In some embodiments the biogas will include about 56 vol. % methane and about 44 vol. % carbon dioxide. In some embodiments the biogas will include about 57 vol. % methane and about 43 vol. % carbon dioxide. In some embodiments the biogas will include about 58 vol. % methane and about 42 vol. % carbon dioxide. In some embodiments the biogas will include about 59 vol. % methane and about 41 vol. % carbon dioxide. In some embodiments the biogas will include about 60 vol. % methane and about 40 vol. vol. % carbon dioxide. In some embodiments the biogas will include about 61 vol. vol. % methane and about 39 vol. vol. % carbon dioxide. In some embodiments the biogas will include about 62 vol. vol. % methane and about 38 vol. % carbon dioxide. In some embodiments the biogas will include about 63 vol. % methane and about 37 vol. % carbon dioxide. In some embodiments the biogas will include about 64 vol. % methane and about 36 vol. % carbon dioxide. In some embodiments the biogas will include about 65 vol. % methane and about 35 vol. % carbon dioxide. In some embodiments the biogas will include about 66 vol. % methane and about 34 vol. % carbon dioxide. In some embodiments the biogas will include about 67 vol. % methane and about 33 vol. % carbon dioxide. In some embodiments the biogas will include about 68 vol. % methane and about 32 vol. % carbon dioxide. In some embodiments the biogas will include about 69 vol. % methane and about 31 vol. % carbon dioxide. In some embodiments the biogas will include about 70 vol. % methane and about 30 vol. % carbon dioxide.

In some embodiments, the biogas 205 is transferred (e.g., with or without further purification) to natural gas-burning boilers (e.g., for upstream processes such as distillation), dryers, or steam generation processes as an energy source, thereby partially or completely replacing natural gas in upstream processes in a dry grind ethanol production process. In some embodiments, the biogas is separated into methane-rich and carbon dioxide-rich portions for downstream applications. Referring to FIG. 4, the separation 207 may be carried out by any suitable means for separating carbon dioxide from methane (e.g., pressure swing adsorption (PSA)). In some embodiments, the biogas is separated into a CO₂-rich stream 212 and a methane-rich stream 210. Purified biogas (e.g., a methane-rich stream or RNG) also may be transferred (e.g., with or without further purification) to natural gas-burning boilers (e.g., for upstream processes such as distillation), dryers, or steam generation processes as an energy source, thereby partially or completely replacing natural gas in upstream processes in a dry grind ethanol production process.

In some embodiments, the CO₂-rich stream 212 comprises CO₂ at a concentration higher than the CO₂ concentration in the biogas. In some embodiments, the CO₂-rich stream comprises CO₂ at a concentration of greater than or equal to 30 vol. %, greater than or equal to 35 vol. %, greater than or equal to 40 vol. %, greater than or equal to 45 vol. %, greater than or equal to 50 vol. %, greater than or equal to 55 vol. %, greater than or equal to 60 vol. %, greater than or equal to 65 vol. %, greater than or equal to 70 vol. %, greater than or equal to 75 vol. %, greater than or equal to 80 vol. %, greater than or equal to 85 vol. %, greater than or equal to 90 vol. %, greater than or equal to 91 vol. %, greater than or equal to 92 vol. %, greater than or equal to 93 vol. %, greater than or equal to 94 vol. %, greater than or equal to 95 vol. %, greater than or equal to 96 vol. %, greater than or equal to 97 vol. %, greater than or equal to 98 vol. %, greater than or equal to 99 vol. %, greater than or equal to 99.5 vol. %, greater than or equal to 99.9 vol. %, or any range or value therein between.

In some embodiments, the CO₂-rich stream may be used as a feedstock in a carbon capture and storage/sequestration (CSS) projects or carbon utilization/sequestration process 213. For example, the CO₂ can be sequestrated in suitable geological formations. In some embodiments, the CCS encompasses the capture and transportation of CO₂ gas to an injection well site where the CO₂ is compressed and injected underground into suitable geologic formations (e.g., Mt. Simon sandstone) to prevent the CO₂ from escaping and re-entering the atmosphere. The CO₂ can also be utilized in gas fermentation, electrolysis, or catalytic CO₂ conversion. These products can be used as fuels, chemical feedstocks, etc. These examples are non-exhaustive and are not intended to be limiting.

In some embodiments, the methane-rich stream 210 comprises methane at a concentration higher than the methane concentration in the biogas. In some embodiments, the methane-rich stream comprises methane at a concentration of greater than or equal to 50 vol. %, greater than or equal to 55 vol. %, greater than or equal to 60 vol. %, greater than or equal to 65 vol. %, greater than or equal to 70 vol. %, greater than or equal to 75 vol. %, greater than or equal to 80 vol. %, greater than or equal to 85 vol. %, greater than or equal to 90 vol. %, greater than or equal to 91 vol. %, greater than or equal to 92 vol. %, greater than or equal to 93 vol. %, greater than or equal to 94 vol. %, greater than or equal to 95 vol. %, greater than or equal to 96 vol. %, greater than or equal to 97 vol. %, greater than or equal to 98 vol. %, greater than or equal to 99 vol. %, greater than or equal to 99.5 vol. %, greater than or equal to 99.9 vol. %, or any range or value therein between.

In some embodiments, the methane-rich stream 210 may be recycled into an upstream process in the dry grind ethanol process. In some embodiments, the methane-rich stream 210 may be used as a heat source for one or more boilers in a dry grind ethanol process. In some embodiments, the methane-rich stream 210 is subjected to further purification 211, which may be one or more further purification steps, to produce a pipeline-quality methane gas 214 or renewable natural gas (RNG).

In some embodiments, the pipeline-quality methane gas or RNG 214 comprises methane at a concentration higher than in the methane-rich stream 210. In some embodiments, the pipeline-quality methane gas comprises methane at a concentration of greater than or equal to 95 vol. %, greater than or equal to 96.0 vol. %, greater than or equal to 96.5 vol. %, greater than or equal to 97.0 vol. %, greater than or equal to 97.5 vol. %, greater than or equal to 98.0 vol. %, greater than or equal to 98.5 vol. %, greater than or equal to 99.0 vol. %, greater than or equal to 99.1 vol. %, greater than or equal to 99.2 vol. %, greater than or equal to 99.3 vol. %, greater than or equal to 99.4 vol. %, greater than or equal to 99.5 vol. %, greater than or equal to 99.6 vol. %, greater than or equal to 99.7 vol. %, greater than or equal to 99.8 vol. %, greater than or equal to 99.9 vol. %, greater than or equal to 99.99 vol. %, or any range or value therein between.

In some embodiments, the biogas is converted to renewable natural gas. In some embodiments, a carbon dioxide removal technology is applied to the biogas to result in concentrated methane. In some embodiments, the purified methane is converted into renewable natural gas. In some embodiments the raw biogas is used as a direct fuel for natural gas-fired boilers. In some embodiments the CO₂ is captured and sequestered or utilized to make other products.

The present technology, thus generally described, will be understood more readily by reference to the following examples, which are provided by way of illustration and are not intended to be limiting of the present technology.

### EXAMPLES

### Example 1. Methane Production Via Anaerobic Fermentation of CTS

To demonstrate the methane production capacity of an anaerobic digester supplied with CTS as a feedstock, an exemplary anaerobic digestion method was demonstrated using CTS obtained as DAF “clears” from a high-efficiency co-product recovery method, such as that disclosed above and in FIGS. 2-4. CTS composition parameters are shown in Table 2 below. Process parameters, including digestion conditions and results, are shown in Table 3 below.

Two reaction conditions were measured; anaerobic digestion of inoculum alone (control), or anaerobic digestion of inoculum plus CTS feedstock (test). CTS was isolated according to the methods described in the present disclosure. In both cases, digestion occurred at 37° C.+/−1 for 28 days. The control digestion had total solids at 32,549 mg/kg and volatile solids at 26,103 mg/kg, with a pH of 7.56. The test digestion had total solids at 46,947 mg/kg and volatile solids at 38,207 mg/kg, with a corrected pH of 6.77.

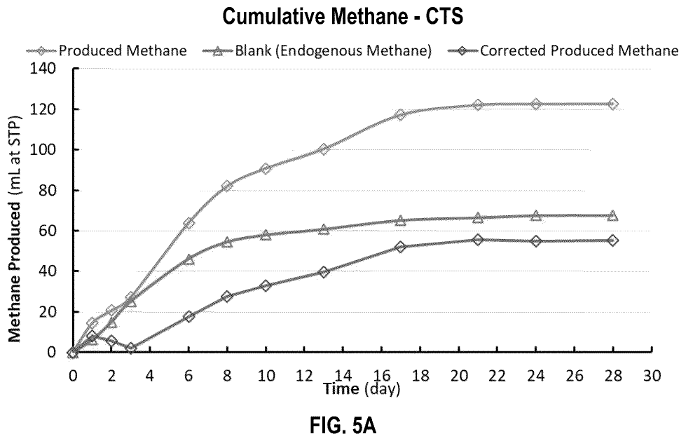

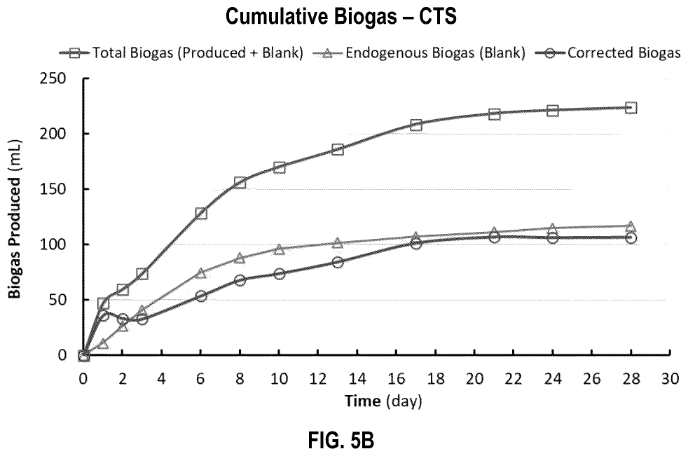

After 28 days, the cumulative biogas was quantified and methane content was determined, as shown in Table 3. Using CTS as feedstock for the anaerobic bioreactor resulted in a substantial increase in methane yield by weight, from 4.5 cubic meters per tonne on inoculum feed alone to 10.53 cubic meters per tonne in the digester fed with clears. These results are also summarized in FIGS. 5A-5B, which show cumulative methane (FIG. 5A) and cumulative biogas (FIG. 5B) produced by methods according to the present disclosure, using CTS as the feedstock for anaerobic digestion.

The results demonstrate the viability of CTS, including CTS produced by the above-discussed high-efficiency co-product recovery methods, as a feedstock for the production of methane via anaerobic digestion.

TABLE 2
Analysis of Feedstock Composition

| | | Feedstock | |
|---|---|---|---|
| Parameter | Method | CTS | CTS Syrup |
| Total Solids (mg/kg) | APHA 2540B | 46,947 | 481,263 |
| Volatile Solids (mg/kg) | APHA 2540E | 38,207 | 401,461 |
| Protein (%) | N × 6.25 | 1.88 | 10.69 |
| Moisture (%) | APHA 2540B | 95.24 | 53.49 |
| Fat (%) | AFGUE-SLM | 0.36 | 3.61 |
| Ash (%) | APHA 2540E | 0.87 | 7.98 |
| Carbohydrates (%) | Calculation | 1.65 | 24.23 |
| Total Organic Carbon | Calculation | 1.91 | 20.07 |
| (%) | | | |
| Total Nitrogen (%) | AFGUE-SLM-0013 | 0.30 | 1.71 |
| Total Sulfur (%) | A221a | 0.06 | 0.52 |
| C:N Ratio | Calculation | 5.66:1 | 10.12:1 |
| BOD (mg/L) | Probe | 20,250 | 202,200 |
| COD (mg/L) | HACH Spec. | 51,900 | 566,750 |
| Alkalinity (mg/L | Titration | 0 | 0 |
| CaCO₃) | | | |
| pH | Probe | 4.47 | 4.25 |

TABLE 3
Analysis of Anaerobic Fermentation of CTS

| Parameter | Method | Feedstock |
|---|---|---|
| Total Solids (mg/kg) | APHA 2540B | 46,947 |
| Volatile Solids (mg/kg) | APHA 2540E | 38,207 |
| Initial pH | Probe | 4.47 |
| Corrected pH | Probe | 6.77 |
| Incubation Temp. (° C.) | — | 37 (±1) |
| Incubation Period (days) | — | 28 |
| Cumulative Biogas (mL) | — | 106.67 |
| Biogas STD Deviation (mL) | — | 1.24 |
| Cumulative Methane (mL) | — | 55.11 |
| Ave. Methane Conc. (%) | — | 69.61 |
| Methane STD Deviation (%) | — | 4.05 |
| Methane Yield (L CH₄/g VS) | — | 0.2755 |
| Methane Yield (m³/metric tonne) | — | 10.53 |

### Example 2. Methane Production Via Anaerobic Fermentation of CTS Syrup

To demonstrate the methane production capacity of an anaerobic digester supplied with CTS syrup as a feedstock, an exemplary anaerobic digestion method was demonstrated using CTS syrup 32a obtained after evaporation of CTS 114, 154, produced by a co-product recovery method as described in U.S. application Ser. No. 16/687,608 (published as US 2020/0154730A1). Syrup composition parameters are shown in Tables 1-2 above. Process parameters, including digestion conditions and results, are shown in Table 4.

Two reaction conditions were measured: anaerobic digestion of inoculum alone (control), or anaerobic digestion of inoculum plus CTS syrup feedstock. CTS syrup was isolated according to the above-discussed high-efficiency co-product recovery methods. In both cases, digestion occurred at 37° C.+/−1 for 28 days. The control digestion had total solids at 32,549 mg/kg and volatile solids at 26,103 mg/kg, with a pH of 7.56. The digestion had total solids at 481,263 mg/kg and volatile solids at 401,461 mg/kg, with a corrected pH of 6.50.

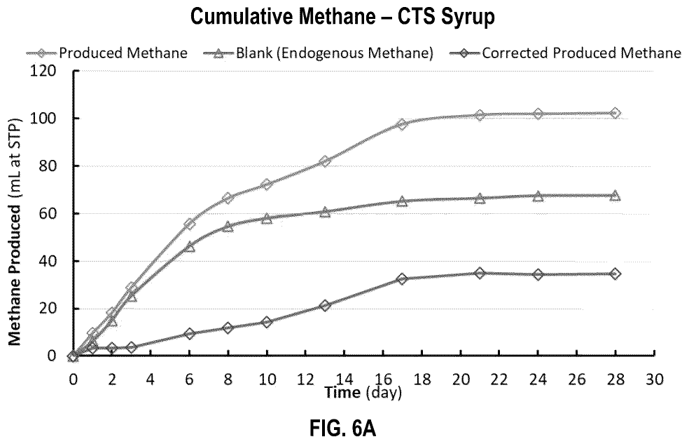

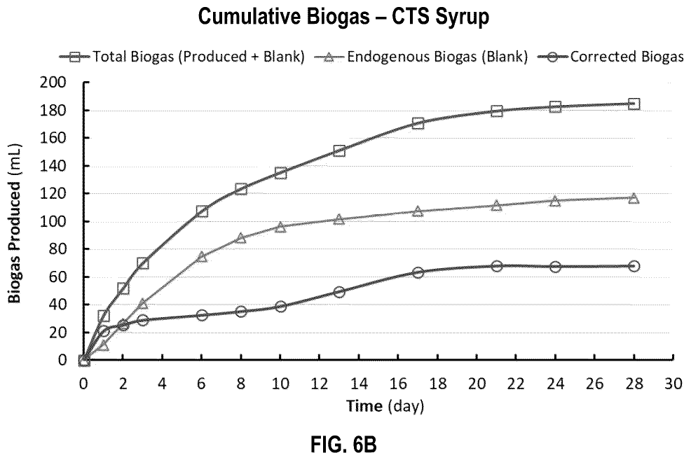

After 28 days, the cumulative biogas was quantified and methane content was determined, as shown in Table 4. Using CTS syrup as feedstock for the anaerobic bioreactor resulted in a substantial increase in methane yield by weight, from 4.5 cubic meters per tonne on inoculum feed alone to 69.64 cubic meters per tonne in the digester fed with syrup. These results are also summarized in FIGS. 6A-6B, which show cumulative methane (FIG. 6A) and cumulative biogas (FIG. 6B) produced by methods according to the present disclosure, using CTS syrup as the feedstock for anaerobic digestion.

The results demonstrate the viability of CTS syrup, including CTS syrup produced by the above-discussed high-efficiency co-product recovery methods, as a feedstock for the production of methane via anaerobic digestion.

TABLE 4
Analysis of Anaerobic Fermentation of CTS Syrup

| Parameter | Method | Feedstock |
|---|---|---|
| Total Solids (mg/kg) | APHA 2540B | 481,263 |
| 26,103 | APHA 2540E | 401,461 |
| Initial pH | Probe | 4.25 |
| Corrected pH | Probe | 6.50 |
| Incubation Temp. (° C.) | — | 37 (±1) |
| Incubation Period (days) | — | 28 |
| Cumulative Biogas (mL) | — | 106.67 |
| Biogas STD Deviation (mL) | — | 1.24 |
| Cumulative Methane (mL) | — | 55.11 |
| Ave. Methane Conc. (%) | — | 69.61 |
| Methane STD Deviation (%) | — | 4.05 |
| Methane Yield (L CH₄/g VS) | — | 0.2755 |
| Methane Yield (m³/metric tonne) | — | 10.53 |

The methods, systems, and compositions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof. It is recognized that various modifications are possible within the scope of the disclosure claimed. Thus, it should be understood that although the present disclosure has been specifically disclosed by preferred embodiments and optional features, modification and variation of the disclosure embodied therein herein disclosed may be resorted to by those skilled in the art, and that such modifications and variations are considered to be within the scope of this disclosure.

The disclosure has been described broadly and generically herein. Each of the narrower species and subgeneric groupings falling within the generic disclosure also form part of the methods. This includes the generic description of the methods with a proviso or negative limitation removing any subject matter from the genus, regardless of whether or not the excised material is specifically recited herein. The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds, compositions, or biological systems, which can, of course, vary. It is also to be understood that the terminology used herein is for the purpose of describing particular embodiments only, and is not intended to be limiting.

One skilled in the art readily appreciates that the present disclosure is well adapted to carry out the objects and obtain the ends and advantages mentioned, as well as those inherent therein. Modifications therein and other uses will occur to those skilled in the art. These modifications are encompassed within the spirit of the disclosure and are defined by the scope of the claims, which set forth non-limiting embodiments of the disclosure.

In addition, where features or aspects of the disclosure are described in terms of Markush groups, those skilled in the art will recognize that the disclosure is also thereby described in terms of any individual member or subgroup of members of the Markush group.

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

### Enumerated Embodiments

1. A method for producing biogas, comprising:
(a) introducing a feedstock comprising clarified thin stillage (CTS) or clarified thin stillage syrup (CTS syrup) to an anaerobic digester; and
(b) digesting the feedstock in the anaerobic digester to produce biogas.

2. The method of embodiment 1, wherein the feedstock comprises CTS.

3. The method of embodiment 1 or embodiment 2, wherein the CTS comprises total solids at a concentration of 3 wt. % to 5 wt. %, relative to the total weight of the CTS.

4. The method of any one of embodiments 1 to 3, wherein the CTS comprises total suspended solids at a concentration of 7 wt. % to 15 wt. %, relative to the total weight of solids in the CTS.

5. The method of any one of embodiments 1 to 4, wherein the feedstock comprises CTS syrup.

6. The method of any one of embodiments 1 to 5, wherein the CTS syrup comprises 10 wt. % to 80 wt. % of total solids, relative to the total weight of the CTS syrup.

7. The method of any one of embodiments 1 to 6, wherein the CTS syrup comprises total suspended solids at a concentration of less than or equal to about 15 wt. %, relative to the total weight of solids in the CTS syrup.

8. The method of any one of embodiments 1 to 7, wherein the CTS syrup comprises total suspended solids at a concentration of 7 wt. % to 15 wt. %, relative to the total weight of solids in the CTS syrup.

9. The method of any one of embodiments 1 to 8, wherein the feedstock comprises CTS and CTS syrup.

10. The method of any one of embodiments 1 to 9, wherein the feedstock further comprises at least one selected from: process condensate; thermal vapor recompression water; water from a CO₂ scrubber unit; or water from CO₂ compression.

11. The method of any one of embodiments 1 to 10, wherein the digesting (b) is performed using a hydraulic retention time of 1 minute to 32 days.

12. The method of embodiment 11, wherein the digesting (b) is performed using a hydraulic retention time of 16 days to 20 days.

13. The method of any one of embodiments 1 to 12, wherein the digesting (b) is performed using a solids retention time of at least 20 days.

14. The method of any one of embodiments 1 to 13, wherein the digesting (b) is performed using a solids retention time of 28 days to 32 days.

15. The method of any one of embodiments 1 to 14, further comprising purifying the biogas to produce renewable natural gas (RNG).

16. The method of embodiment 15, wherein the purifying comprises separating CO₂ from the biogas to produce a CO₂-rich stream and a methane-rich stream.

17. The method of embodiment 16, wherein the CO₂-rich stream is further directed to a carbon sequestration or utilization process.

18. The method of embodiment 16 or embodiment 17, wherein the methane-rich stream is further purified to produce the renewable natural gas (RNG).

19. The method of any one of embodiments 15 to 18, further comprising blending the RNG with natural gas.

20. The method of any one of embodiments 1 to 19, wherein the anaerobic digester is a high-rate reactor.

21. The method of any one of embodiments 1 to 20, wherein the anaerobic digester is a stirred tank reactor.

22. The method of any one of embodiments 1 to 21, wherein the CTS or CTS syrup has a native C:N ratio.

23. The method of any one of embodiments 1 to 22, wherein an additive ingredient is added to the CTS or the CTS syrup to modify a C:N ratio.

24. The method of any one of embodiments 1 to 23, further comprising removing residual biomass remaining in the anaerobic digester after (b).

25. The method of any one of embodiments 1 to 24, further comprising processing residual biomass remaining in the digester after (b) for use as a fertilizer.

26. The method of any one of embodiments 1 to 25, further comprising processing residual biomass remaining in the digester after (b) for use as single cell protein.

27. The method of any one of embodiments 1 to 26, further comprising directing the biogas, methane-rich gas stream, or RNG to an upstream process as an energy source.

28. A method of producing renewable natural gas (RNG), the method comprising:
(a) introducing a feedstock comprising clarified thin stillage (CTS) or clarified thin stillage syrup (CTS syrup) to an anaerobic digester;
(b) digesting the feedstock to produce biogas;
(c) removing CO₂ from the biogas to produce a CO₂-rich stream and a methane-rich stream; and
(d) further purifying the methane-rich stream to produce renewable natural gas.

29. The method of embodiment 28, wherein the feedstock comprises CTS.

30. The method of embodiment 28 or embodiment 29, wherein the CTS comprises total solids at a concentration of 3 wt. % to 5 wt. %, relative to the total weight of the CTS.

31. The method of any one of embodiments 28 to 30, wherein the CTS comprises total suspended solids at a concentration of 7 wt. % to 15 wt. %, relative to the total weight of solids in the CTS.

32. The method of any one of embodiments 28 to 31, wherein the feedstock comprises CTS syrup.

33. The method of any one of embodiments 28 to 32, wherein the CTS syrup comprises 10 wt. % to 80 wt. % of total solids, relative to the total weight of the CTS syrup.

34. The method of any one of embodiments 28 to 33, wherein the CTS syrup comprises total suspended solids at a concentration of less than or equal to about 15 wt. %, relative to the total weight of solids in the CTS syrup.

35. The method of any one of embodiments 28 to 34, wherein the CTS syrup comprises total suspended solids at a concentration of 7 wt. % to 15 wt. %, relative to the total weight of solids in the CTS syrup.

36. The method of any one of embodiments 28 to 35, wherein the feedstock comprises CTS and CTS syrup.

37. The method of any one of embodiments 28 to 36, wherein the feedstock further comprises at least one selected from: process condensate; thermal vapor recompression water; water from a CO₂ scrubber unit; or water from CO₂ compression.

38. The method of any one of embodiments 28 to 37, wherein the digesting (b) is performed using a hydraulic retention time of 1 minute to 32 days.

39. The method of embodiment 38, wherein the digesting (b) is performed using a hydraulic retention time of 16 days to 20 days.

40. The method of any one of embodiments 28 to 39, wherein the digesting (b) is performed using a solids retention time of at least 20 days.

41. The method of any one of embodiments 28 to 40, wherein the digesting (b) is performed using a solids retention time of 28 days to 32 days.

42. The method of any one of embodiments 28 to 41, wherein the CO₂-rich stream is further directed to a carbon capture process or sequestration process.

43. The method of any one of embodiments 28 to 42, further comprising blending the RNG with natural gas.

44. The method of any one of embodiments 28 to 43, wherein the anaerobic digester is a high-rate reactor.

45. The method of any one of embodiments 28 to 44, wherein the anaerobic digester is a stirred tank reactor.

46. The method of any one of embodiments 28 to 45, wherein the CTS or CTS syrup has a native C:N ratio.

47. The method of any one of embodiments 28 to 46, wherein an additive ingredient is added to the CTS or the CTS syrup to modify a C:N ratio.

48. The method of any one of embodiments 28 to 47, further comprising removing residual biomass remaining in the anaerobic digester after (b).

49. The method of any one of embodiments 28 to 48, further comprising processing residual biomass remaining in the digester after (b) for use as a fertilizer.

50. The method of any one of embodiments 28 to 49, further comprising processing residual biomass remaining in the digester after (b) for use as single cell protein.

51. The method of any one of embodiments 28 to 50, further comprising directing the biogas, methane-rich gas stream, or RNG to an upstream process as an energy source.

What is claimed is:

1. A method for producing biogas, comprising:
(a) introducing a feedstock comprising clarified thin stillage (CTS) or clarified thin stillage syrup (CTS syrup) to an anaerobic digester; and
(b) digesting the feedstock in the anaerobic digester to produce biogas.

2. The method of claim 1, wherein the feedstock comprises CTS.

3. The method of claim 1, wherein the CTS comprises total solids at a concentration of 3 wt. % to 5 wt. %, relative to the total weight of the CTS.

4. The method of claim 1, wherein the CTS comprises total suspended solids at a concentration of 7 wt. % to 15 wt. %, relative to the total weight of solids in the CTS.

5. The method of claim 1, wherein the feedstock comprises CTS syrup.

6. The method of claim 1, wherein the CTS syrup comprises 10 wt. % to 80 wt. % of total solids, relative to the total weight of the CTS syrup.

7. The method of claim 1, wherein the CTS syrup comprises total suspended solids at a concentration of less than or equal to about 15 wt. %, relative to the total weight of solids in the CTS syrup.

8. The method of claim 1, wherein the CTS syrup comprises total suspended solids at a concentration of 7 wt. % to 15 wt. %, relative to the total weight of solids in the CTS syrup.

9. The method of claim 1, wherein the feedstock comprises CTS and CTS syrup.

10. The method of claim 1, wherein the feedstock further comprises at least one selected from: process condensate; thermal vapor recompression water; water from a CO₂ scrubber unit; or water from CO₂ compression.

11. The method of claim 1, wherein the digesting (b) is performed using a hydraulic retention time of 1 minute to 32 days.

12. The method of claim 1, wherein the digesting (b) is performed using a solids retention time of at least 20 days.

13. The method of claim 1, further comprising purifying the biogas to produce renewable natural gas (RNG).

14. The method of claim 13, wherein the purifying comprises separating CO₂ from the biogas to produce a CO₂-rich stream and a methane-rich stream.

15. The method of claim 14, wherein the CO₂-rich stream is further directed to a carbon capture process or sequestration process.

16. The method of claim 14, wherein the methane-rich stream is further purified to produce the renewable natural gas (RNG).

17. The method of claim 13, further comprising blending the RNG with natural gas.

18. The method of claim 1, wherein the anaerobic digester is a high-rate reactor or a stirred tank reactor.

19. The method of claim 1, wherein:
the CTS or CTS syrup has a native C:N ratio; or
an additive ingredient is added to the CTS or the CTS syrup to modify a C:N ratio.

20. The method of claim 1, further comprising at least one of:
removing residual biomass remaining in the anaerobic digester after (b);
processing residual biomass remaining in the digester after (b) for use as a fertilizer;
processing residual biomass remaining in the digester after (b) for use as single cell protein; or
directing biogas, a methane-rich gas stream, or RNG to an upstream process as an energy source.
